(19) United States
(12) Patent Application Publication
(10) Pub. No.: US 2014/0316361 A1
(43) Pub. Date: Oct. 23, 2014

(54) PROCESS FOR THE MANUFACTURE OF A DRESSING AND DRESSING OBTAINED

(71) Applicant: Christian Stephenson, Wilmslow (GB)

(72) Inventor: Christian Stephenson, Wilmslow (GB)

(73) Assignee: Medtrade Products Ltd., Crew Chesire (GB)

(21) Appl. No.: 13/979,169

(22) PCT Filed: Jan. 11, 2012
(86) PCT No.: PCT/EP2012/050376

§ 371 Date: Sep. 24, 2013

(30) Foreign Application Priority Data
Jan. 11, 2011 (GB) 1100426.4

(51) Int. Cl. A61L 15/28

(52) U.S. Cl. CPC A61L 15/28
USPC 604/367; 536/20

(57) ABSTRACT

A process for producing a salt of a chitosan acid anion derivative, which comprises contacting chitosan fibre (a) with a base in an aqueous or non-aqueous medium and (b) with a solution in an aqueous or non-aqueous medium of a salt of the formula (I): M-R-T wherein M is a Group IA metal cation; R is a hydrocarbyl oxoacid anion, substituted by T; and T is a nucleophilic leaving group, all at a temperature below 50° C., preferably in the range of 30 to 40° C.

The present invention relates to an anti-microbial component of a absorbent device, to a method for the preparation of such an component, and in particular to a method for the preparation of a novel hydrophilic, anti-microbial derivatised chitosan, including in the form or fibres, and a method for applying metal ions to the chitosan, and also to an absorbent device comprising the component, a method for the preparation of such an absorbent device, and the use of said absorbent device.

The term ‘absorbent medical device’ as used herein includes wound dressings for acute wounds, including surgical wounds, and chronic and burn wounds, ostomy devices, surgical and dental sponges, and absorbent pads for the personal care sector, particularly for disposable sanitary devices such as nappies (diapers), disposable nappies and training pants, feminine care products, for example, tampons, sanitary towels, or napkins and pant liners, and incontinence products. It in particular includes (but is not limited to) an absorbent medical device comprising as an absorbent material, a water-insoluble organic or inorganic acyl substituted hydrocarbyl chitosan.

Reference to a “derivatised chitosan” herein is to a product of the process described herein, which is believed to be a chitosan in which at least a proportion of the chitosan hydroxyl functions have been converted to salified hydroxyl-(organic or inorganic acyl)-hydrocarbyloxy groups.

The term ‘organic or inorganic acyl’ as used herein includes organic acid residues such as carboxyl and inorganic acid residues such as sulphonyl.

By “substantially insoluble” herein is meant that when a material, for example a derivatised chitosan is exposed to an excess of an aqueous medium it does not dissolve into solution, or at least that dissolution is so low as to have no significant effect on the physical properties of the polymer.

Reference to the “absorbency” of a derivatised chitosan or a product such as a non-woven fabric consisting essentially of the fibres herein is to the capacity of the derivatised chitosan or product to take up fluid.

In the case of the present derivatised chitosan fibres, fluid is absorbed into the internal fibre structure and the fibre swells. In the case of a non-woven fabric consisting essentially of the fibres, the overall absorptive capacity is sensitive to the sizes and interconnectivity of the inter-fibre volumes within the fabric, and hence its process for manufacture. However, in the case of the present fibres and non-woven fabrics, the absorbencies will not differ greatly.

Measurement of the overall absorptive capacity of an absorbent material or of a absorbent device, in particular a medical absorbent device, comprising such material is a convenient and effective process for determining the effectiveness of the absorbent material or device for absorbent applications, such as wound dressings.

Wound-contacting materials in dressings should preferably have good absorbency whilst being substantially insoluble, should maintain their integrity when wet, not adhere to the wound, and have antimicrobial properties.

For a long time, wound dressings for acute wounds, including surgical wounds, have been sterile wadding and cotton-based gauze, the use of which suffers from certain limitations.

Cotton gauze does not possess any anti-microbial characteristics. Although applied as a sterile dressing, cotton gauzes can be infected by microbes in use. These traditional dressings tend to adhere to a wound and even be integrated into new soft tissue, causing pain and new wounds to the patient during a change of dressing. The remaining fragments of these traditional dressings on the wound bed after a change of dressing can adversely affect the healing of the wound. Other dressings made of synthetic materials also have the same drawbacks.

When treating chronic wounds, a wound dressing is desirable to absorb a high volume of exudate and at the same time promote the healing of the wound. The currently widely used alginate fibre dressings do not have enough absorption ability to treat a wound exuding a high volume of exudate.

Polyacetylglucosamine, also known as chitin, is an amylose widely existing in nature. It is a major composition of fungal cell wall and carapace of shrimps, crabs and insects. It may be at least partially deacetylated to produce free hydroxyl functions, and so to form chitosans with varying degrees of deacetylation and free hydroxyl functions.

It will be appreciated that the composition of any chitosan may vary with the source, the degree of deacetylation and the molecular weight of the chitin starting material that is used in its production,

A chitosan has a unique property of being absorbed by human tissue after hydrolysis by lysozyme. This material is non-toxic, odourless and compatible with human tissue and does not cause any immune response. It also has antibiotic, antiphlogistic, haemostatic and antalgic capability, and facilitates wound healing.

US 2005/0058694 mentions a component of a wound dressing partially composed of an amine acid addition salt of a partially carboxymethylated chitosan. The carboxymethylation is achieved by one-step process for treating chitosan fibres with a water/alcohol solution containing sodium chloroacetate and sodium hydroxide at the boiling point of the reaction mixture, i.e. at in excess of 78° C. The partially sodium carboxymethylated, partially alkalised chitosan product is washed with acidulated ethanol to convert it to an amine acid addition salt. It is not clear whether the sodium carboxymethyl groups and alkalised hydroxyl groups are converted to carboxymethyl groups and hydroxyl groups respectively.

The end-product modified chitosan fibre is air-dried and processed by a non-woven processing machine, into non-woven fabric after fibre opening, web formation and needling. The chitosan product in the dressing that is so obtained becomes a semi-transparent gel material in the presence of water and its fibre structure is visible.

The first technical problem of the prior art is that the high aqueous absorbency of partially carboxymethylated chitosan fibre (optionally as an amine salt) causes difficulty in fibre opening and web formation during subsequent processing of fibre to a non-woven fabric by a non-woven technique.

The second technical problem of the prior art is that after being processed by a non-woven processing machine, the fibre finish on the surface of the fibre makes the non-woven fabric hydrophobic.

The hydrophobicity adversely affects the fluid absorbency of the dressings of the prior art, in particular from acute wounds, including surgical wounds, and chronic and burn wounds.

To be useful in an absorbent device, in particular a wound dressing, the fibres of an absorbent material suitably have an absorbency of 8 to 30 grams per gram (g/g) standard solution, preferably 12 to 27 g/g, and more preferably 16 to 23 g/g.

It is therefore an object of the present invention to provide a therapeutically active derivatised chitosan fibre which is readily processable in fibre opening and web formation during subsequent processing to a non-woven fabric component of such a wound dressing, thus overcoming the corresponding disadvantage of the prior art materials.

It is also an object of the present invention to overcome the disadvantage of the hydrophobicity of prior art fabrics, and to provide a therapeutically active derivatised chitosan fibre for a dressing component which has a wound-facing surface which is not hydrophobic and has good absorbency of fluids, in particular from acute wounds, including surgical wounds, and chronic and burn wounds, and which also meets the above criteria for adequate and good absorbency of fluids, in absorbent devices, and in particular in advanced wound dressings.

We have also found that when such a carboxymethylated chitosan and its congeners are produced at temperatures above 45° C., an undesirable side reaction starts to occur, which impairs the above desirable qualities of processable fibres and absorbent devices produced from them.

It is not necessary to use temperatures as low as 20° C. as in the prior art to avoid degradation of the chitosan in the reaction, and when produced at temperatures below 25° C., an inferior product is produced, and the process is uneconomically slow.

In order to solve the above technical problems, according to a first aspect of the present invention, there is provided a process for producing a salt of a chitosan acid anion derivative, which comprises in either order contacting chitosan fibre
a) with a base in an aqueous or non-aqueous medium and
b) with a solution in an aqueous or non-aqueous medium of a salt of the formula (I):

M-R-T (I)

wherein
M is a Group IA metal cation;
R is a hydrocarbyl oxoacid anion, substituted by T; and
T is a nucleophilic leaving group,
   all at a temperature below 50° C.

it is greatly preferred that the product should not subsequently be washed or otherwise treated with acid or acid materials, in order to avoid the formation of the undesirable products of the prior art.

Without limiting the invention in any way, the derivatised chitosan fibre product from this process is believed to be one in which one or both of the two types of hydroxyl groups on the D-glucosamine units of the chitosan are randomly converted at least in part to M-R—O— groups (etherification), the other generally being converted to a hydroxyl group derivatised by the base.

The base is preferably an alkali metal hydroxide, in which case the metal cation is preferably the same as M in the compound of formula (I), preferably an alkali metal hydroxide, such as sodium hydroxide, and the other hydroxyl group on the D-glucosamine units of the chitosan are converted to M-O— groups (alkalisation).

Either substitution may take place at either hydroxyl position in the glucosamine units of the chitosan macromolecule, in any distribution up to the maximum degree of substitution that is possible. The average degree of substitution refers to the mean number of hydroxyl groups in all positions converted to M-R—O— groups, that is, the mean number of moles of M-R—O— groups per mole of D-glucosamine unit in the chitosan polymer.

The maximum degree of substitution in a chitin which has been 100% deacetylated is therefore 2, when each D-glucosamine unit is substituted at both hydroxyl positions, and the degree of substitution when an average of one hydroxyl group is substituted per D-glucosamine unit is 1.

It will be appreciated that the maximum possible degree of the above substitution will depend on the proportion of free deacetylated hydroxyl groups in the chitosan available for substitution. The degree of deacetylation is often from 60 to 100%.

However, the average degree of substitution is suitably less than 0.8, preferably less than 0.7, more preferably less than 0.6 for the derivatised chitosan to be substantially water-insoluble. The average degree of substitution in the derivatised chitosan polymer of the present invention may more suitably be from about 0.1 to about 0.4, for example from about 0.15 to about 0.35, such as from about 0.2 to 0.3.

The average molecular weight of the starting material chitosan is often between 3800 to 20,000 daltons.

It will be appreciated that the composition of the starting material chitosan may vary with its source, its average degree of substitution will depend in part on the degree of deacetylation of the chitin starting material that is used in its production, and its molecular weight will depend on that of the chitin. For example, a natural chitosan derived from shrimp or snow crab and/or a natural chitin may be utilised.

A chemically modified chitosan or an unmodified or modified related material, such as a modified chitin, may also be used as a starting material in the present process, provided it contains substitutable glucosamine OH groups or groups convertible thereto. All of these materials may be modified in the present process to produce absorbent products, for example for use in wound care medical devices.

The other reagents, preferably in aqueous solution, may be brought into contact with the chitosan by methods known in the art, for instance preferably by mixing using stirrers, or by spraying onto the chitosan in sufficient volume as noted below.

The process is an alkalisation and etherification process, which may also be carried out as a single step in which the base and the compound of formula (I) are added at the same time in one reaction vessel (a “one-pot” process).

The one pot process is desirable because it is easier and quicker than a two-sub-step process step, and by minimising the number of reaction steps a higher yield may be obtained.

When the alkalisation and etherification sub-steps are simultaneous in a one-pot process to produce a chitosan derivative of the present invention, the reaction rate is often higher than that observed for the equivalent reaction at the same temperature in which alkalisation and etherification are carried out in separate steps.

A one-pot process also minimises the time of exposure of the chitosan to the base, therefore keeping the alkaline, oxidative degeneration of the chitosan to a minimum. It is necessary to minimise degeneration of the chitosan during processing in order to ensure that the modified chitosan is sufficiently strong to be useful as an absorbent material in a wound dressing, and indeed to maximise both the dry strength and the wet strength of the product.

If the level of water used in the reaction is reduced, the reaction rate increases and the fibre strength increases to a useable level. However, a certain amount of diluent is required to be practicable, particularly in wetting the chitosan and ensuring an even and complete reaction, and again to lower the concentration of the base to a level that minimizes any degradation of the chitosan.

Thus, according to an embodiment of this first aspect of the present invention, there is also provided a one-step process for producing a salt of a chitosan acid anion derivative, which comprises simultaneously contacting chitosan fibre
a) with a base in an aqueous or non-aqueous medium and
b) with a solution in an aqueous or non-aqueous medium of a salt of the formula (I):

M-R-T (I)

wherein
M is a Group IA metal cation;
R is a hydrocarbyl oxoacid anion, substituted by T; and
T is a nucleophilic leaving group,
   all at a temperature below 50° C.

However, less preferably, alkalisation and etherification may be carried out in two separate reaction steps, treating the chitosan first with a base and then the compound of formula (I), or vice versa.

Preferably alkalisation and etherification (preferably effected in a single reaction step) are carried out in an aqueous medium. Most preferably alkalisation and etherification are carried out in a mixture of water and a miscible or soluble organic solvent, in which the base and the compound of formula (I) are soluble, such as an alcohol, for example methanol, iso-propanol, or preferably ethanol.

As noted above, the base is preferably an alkali metal hydroxide, in which case the metal cation is preferably the same as M in the compound of formula (I), preferably an alkali metal hydroxide such as sodium hydroxide.

Generally, the higher the concentration of given base, the faster the rate of reaction. The nature and concentration of the base should be balanced with the nature and concentration of the compound of formula (I) and need to avoid degradation of the chitosan. Where the base is sodium hydroxide, a solution with a percentage concentration with respect to the total reaction mixture of 2.5 to 12.5, for example 2.5 to 10 w/v % has been found to be suitable.

The chitosan may be used in the form of fibre tow in the reaction for the substitution of chitosan with M-R groups. The fibres may be used in a wide range of lengths, for example a few mm, such as 2 mm to 5 mm, to several tens of mm, for 100 mm or more. It will be clear to those skilled in the art that the physical form of the chitosan starting material may have a significant effect on the physical form of the derivative chitosan product of the process.

M may suitably be a sodium or potassium cation, preferably a sodium cation.

R may suitably be a hydrocarbyl acylate of an organic or inorganic oxoacid substituted by a nucleophilic leaving group T, for example an alkane acylate of an organic or inorganic oxoacid, substituted by a nucleophilic leaving group T.

R may thus suitably be, for example, an alkanoate, preferably a lower alkanoate with 2 to 6 carbon atoms, such as an acetate, glyoxylate, propionate, pyruvate or butyrate, preferably acetate, propionate or butyrate preferably substituted by a nucleophilic leaving group T in the 1-position. The alkanoate moiety may be branched or unbranched, and hence suitable butyrates may be n-butyrate or iso-butyrate. The alkanoate group that is most preferred is acetate, preferably substituted by a nucleophilic leaving group T in the 2-position.

R may also suitably be, for example an alkanesulphonate, preferably a lower alkanesulphonate with 2 to 6 carbon atoms, such as a methanesulphonate, ethanesulphonate, or propanesulphonate, preferably substituted by a nucleophilic leaving group T in the 1-position, and more preferably methanesulphonate, substituted by a nucleophilic leaving group T in the 1-position. The alkane moiety may be branched or unbranched, and hence suitable propane sulphonates may be propane-1- or 2-sulphonate, and butanesulphonates may be butane-1-sulphonate, 2,2-dimethylethane-1-sulphonate or 1,2-dimethylethane-1-sulphonate.

The alkane sulphonate substituent group that is most preferred is ethane sulphonate, preferably substituted by a nucleophilic leaving group T in the 2-position.

The nucleophilic leaving group T, when R is an alkanoate, may suitably be —OSO₂CF₃ triflate, —OSO₂F fluorosulfonate, —OTs tosylate, —OMs mesylate, —I iodide, —Br bromide, —Cl chloride, —ONO₂ nitrate, —OPO(OH)₂ phosphate, and other inorganic esters, —F fluoride, each preferably in the 2-position, and more preferably is chloro on acetate.

The nucleophilic leaving group T, when R is an alkanesulphonate, may suitably be —OSO₂CF₃ triflate, —OSO₂F fluorosulfonate, —OTs tosylate, —OMs mesylate, —I iodide, —Br bromide, —Cl chloride, —ONO₂ nitrate, —OPO(OH)₂ phosphate, and other inorganic esters, —F fluoride, preferably in the 1-position, and more preferably is chloro on methanesulphonate.

R may also suitably be an arenecarboxylate, such as a benzoate or toluate, substituted by a nucleophilic leaving group T.

R may also suitably be an arenesulphonate, such as benzenesulphonate or toluenesulphonate, substituted by a nucleophilic leaving group T.

Suitable and preferred nucleophilic leaving groups T, when R is an arenecarboxylate or an arenesulphonate, and suitable and preferred substitution positions for T will be well-known to the skilled person.

The amount of water in the reaction mixture may also affect the reaction rate. Lowering the water content in a reaction in which alkalisation and etherification are carried out simultaneously results in a significant increase in reaction rate. Lowering the water content in the etherification (by base) step of a reaction in which alkalisation and etherification are carried out separately increases the rate, but to a lesser extent. In all cases however it is preferred that the concentration of the base is kept below the figures given hereinbefore to avoid deleterious effects on the chitosan.

As regards the reaction temperature, generally, the higher the reaction temperature, up to 50° C., the faster the rate of reaction. The elevation of the temperature, however, should be balanced with the nature and concentration of the compound of formula (I) and the need to avoid the formation of the disadvantageous partially carboxymethylated chitosan amine salt fibres of the prior art, which are formed at or near the boiling point of the reaction mixture, or beyond it if a pressurised system is used, and indeed start to be formed above 45° C.

(As noted above, the prior art fibres have a high aqueous absorbency, which causes difficulty in fibre opening and web formation during subsequent processing of the prior art derivatised chitosan fibre to a non-woven fabric by a non-woven technique, and after being processed by a non-woven processing machine, the fibre finish on the surface of the partially carboxymethylated chitosan fibre makes the non-woven fabric hydrophobic. The hydrophobicity adversely affects the fluid absorbency of the dressings of the prior art.)

Equally, as noted above, a reaction rate at room temperature, say 20° C., is uneconomically slow, and is unnecessary to avoid oxidative degradation of the chitosan by the base in the reaction.

Thus, as regards the reaction temperature, for the process overall it may suitably be 25 to 45° C. For example, about 30 to 40° C. has been found to be suitable to give a useful degree of substitution in an economic time, without deleterious side reactions, and with a product of consequently improved physical properties. The reaction time for the process overall to give a useful degree of substitution in an economic time may suitably be 0.5 to 8 hours, for example 1 to 5 hours, such as 1 to 3 hours.

Further, fresh charges of reactant can be introduced at any time throughout the reaction. The degree of substitution can be controlled in particular by control of the reaction time and temperature.

After the reaction, the remaining solution is removed, and the product can then be washed free of by-products and impurities by employing washing stages known in the art.

Such stages include washing with water, organic liquids, or mixtures thereof. Particularly useful are mixtures of a lower alcohol and water. Washing efficiency can be enhanced by washing at temperatures up to 50° C.

As noted above, it is greatly preferred that the product should not subsequently be washed or otherwise treated with acid or acid materials, in order to avoid the formation of the undesirable products of the prior art.

Finally the derivatised chitosan should be dried to remove residual liquid from the previous stages. Drying can be carried out by methods known in the art such as forced air drying, radiant heat drying etc.

The simplicity of the chemistry and the availability of the reactants enable the cost of manufacture of medical and other devices (as hereinbefore defined) to be kept advantageously low.

The derivatised chitosans of the invention are highly advantageous for use as absorbent materials in absorbent devices because they are non-toxic, odourless and compatible with human tissue and do not cause any immune response. They also have anti-microbial, antiphlogistic, haemostatic and antalgic capability, and facilitate wound healing.

The therapeutically active, absorbent derivatised chitosan fibres of the present invention meet the criteria for adequate and good absorbency of fluids in absorbent devices, and in particular in advanced wound dressings. The derivatised chitosan fibres have an absorbency of 8 to 30 grams per gram (g/g) of saline solution, often 12 to 27 g/g, and more often 16 to 23 g/g.

The dry strength of the derivatised fibres must be sufficient to enable processing into woven or preferably non-woven structures, and, to be useful as an absorbent material in an absorbent device, in particular a wound dressing, the wet strength of the material must be sufficient to allow removal from the site in one piece.

The pre-absorption derivatised chitosan fibres according to the present invention may have a monofilament linear density of 0.1 to 30, preferably about 0.5 to 20, and more preferably 0.9 to 4, for example 1 3 to 5 decitex, and a strength of 0.8 to 2.2, such as 1 to 2, for example 1.2 to 1.8 cN/dtex.

Whilst they swell in contact with water to become an elastic gel material, and exhibit good absorption and retention of fluid, they maintain their integrity sufficiently, for example in wound dressings, to be removed from the wound site in one piece, without irrigation, and with minimum pain and shedding. Absorption of fluid is virtually instantaneous since ionic exchange is not required for the fibres to become gellable.

However, the initial aqueous absorbency of the derivatised chitosan fibre is not so high that it causes difficulty in fibre opening and web formation during subsequent processing of the derivatised chitosan fibre to a non-woven fabric by a non-woven technique.

Moreover, the water-insoluble inorganic or organic acyl substituted hydrocarbyl chitosans of the present invention are also advantageous compared to the prior art carboxymethyl chitosans because the absorptive capacity is not adversely affected by processing into absorbent device, for example wound dressing, non-woven components. They are also affected to a lesser extent in use by changes in pH. Wound dressings containing these materials continue to absorb to a good level at low pH.

The inorganic or organic acyl substituted hydrocarbyl chitosans of the present invention may be processed according to known methods into a wide variety of forms, before being processed into a component of an absorbent device by methods described further hereinafter. The manner in which the derivative chitosan is processed has a significant effect on the properties of the final product, particularly the strength, gelling time, and absorbency.

For example, as noted above, the water-insoluble inorganic or organic acyl substituted hydrocarbyl chitosan according to the invention has anti-microbial properties.

This therapeutic property of the derivatised chitosan according to the present invention may be enhanced by introducing to the derivatised chitosan a non-toxic, therapeutically effective amount, which is also compatible with human tissue and does not cause any immune response, of metal cations with anti-microbial properties, which are subsequently released in an aqueous environment, such as in wound fluid, in an efficacious manner. The use of different concentration of cations enables the release profile of the chitosan to be tailored as desired.

Suitable cations include Group IB and Group IIB metal cations, such as copper, zinc and, preferably silver ions into the derivatised chitosan amine salt by exchanging at least part of the M alkali metal cations in the chitosan with silver I cations, such that after the application of the silver salt, sufficient silver cations are ionically bonded to form a new silver salt with the derivatised chitosan, that the latter is antimicrobially effective.

The process for doing so may be effected, for example in the case of silver cations, by contacting the washed and dried derivatised chitosan fibre with a source of silver I cations. Appropriate sources of silver include salts that are soluble in water and/or an organic solvent, in which the derivatised chitosan is insoluble.

Examples include as water or an optionally aqueous alcohol, for example methanol, iso-propanol, or preferably ethanol. Such silver salts include silver nitrate, silver sulphate, silver lactate, silver acetate, silver citrate, and/or mixtures thereof. Generally, the higher the solubility of a given salt, the higher the potential maximum concentration of silver ions in solution, and hence in the derivatised chitosan.

The presence of water and the aqueous absorbency of the derivatised chitosan fibres may cause a technical problem of premature gelling of the fibre by aqueous solutions. Thus the solution should preferably contain no or a minimal water component, or a minimum quantity of liquid is applied in a finely dispersed form, such as by spraying an aerosolised solution of silver salt at a high concentration

Preferably, silver nitrate in ethanol solvent at a concentration of silver nitrate of 0.5 to 10 w/v % is added. As regards the reaction temperature, for the process overall it may suitably be 20 to 30° C. This has been found to be suitable to give a useful degree of ion exchange in an economic time. The reaction time for the process overall may suitably be 0.25 to 4 hours. For example, 0.5 to 2 hr has been found to be suitable to give a useful degree of ion exchange in an economic time.

For any of the derivatised chitosans described hereinbefore, after the relevant formative reaction the product is usually washed free of by-products and impurities by employing washing stages known in the art.

Such stages include washing with organic liquids, or mixtures thereof. Lower alcohols, such as ethanol, or mixtures thereof are particularly useful. Washing efficiency can be enhanced by washing at temperatures up to 50° C.

Finally the derivatised chitosan should be dried to remove residual liquid from the previous stages. Drying can be carried out by methods known in the art such as forced air drying, radiant heat drying etc.

The simplicity of the chemistry and the availability of the reactants enable the cost of manufacture of medical and other devices (as hereinbefore defined) to be kept advantageously low.

The inorganic or organic acyl substituted hydrocarbyl chitosans produced by the process of the first aspect of the present invention may be processed according to known methods into a wide variety of forms, depending on their intended use. The manner in which the derivative chitosan is processed has a significant effect on the properties of the final product, particularly the strength, gelling time, and absorbency.

In many embodiments of absorbent devices comprising the water-insoluble inorganic or organic acyl substituted hydrocarbyl chitosan according to the invention, it is the only absorbent material present. Such embodiments do not contain other absorbent materials such as hydrogels, anion-exchange resins, etc. or relatively non-absorbent, for example strengthening polymeric, materials

In other embodiments of absorbent devices comprising the water-insoluble derivatised chitosan according to the invention, however, other materials are present. Such embodiments may contain other absorbent materials such as hydrogels, anion-exchange resins, etc. or relatively non-absorbent, for example strengthening polymeric materials, often in the form of fibres which are intermingled with the derivatised chitosan according to the invention.

In another embodiment, the present invention is directed to an absorbent medical device comprising derivatised chitosan of the present invention which is reinforced with a reinforcing fibre blended with the water-insoluble derivatised chitosan.

Examples of other materials that may be present include other absorbent materials, such as hydrogels, for example an alginate, or anion-exchange resins, or a mixture thereof; or relatively non-absorbent, for example polymeric strengthening, materials, such as unmodified chitosan or thermoplastic bicomponent fibres, most preferably having a polyolefin component, for example comprising a polyolefin-containing polymeric material of which the largest part (by weight) consists of homo- or copolymers of monoolefins such as ethylene, propylene, 1-butene, 4-methyl-l-pentene, etc.

Preferably, such materials are added after drying. For example, un-modified chitosan fibre may be added, in a weight ratio of chitosan fibre to the derivatised chitosan fibre of 1:9 to 9:1, for example of 1:6 to 6:1 or 1:3 to 3:1. It will be clear to those skilled in the art that the addition of unmodified chitosan to the derivatised chitosan material may have a significant improving effect on the strength of the material in a non-woven wound dressing.

The air-dried derivatised chitosan fibre (including any such additive materials) may then be converted into a non-woven fabric by fibre opening, web formation and needling, which may suitably be of 30 to 200 g/m2 or more. Such a non-woven fabric forms a second aspect of the present invention.

It will be clear to those skilled in the art that the area density of the processed derivative chitosan material may have a significant effect on the total absorbency of the product of the processing.

The non-woven fabric may then be converted to an anti-microbial absorptive component of a non-woven absorptive device by cutting, packaging and sterilising.

Thus, in a third aspect of the present invention, an absorbent device (as defined hereinbefore) comprising the derivatised chitosan fibres is provided.

Their absorbent properties, biodegradability, and the fact that chitosan is a renewable material, mean that the derivatised chitosan absorbent materials of the present invention may be used in a wide range of absorbent devices.

The absorbent materials of the present invention exhibit instant gelling in aqueous media, good absorbency and, crucially, good retention of absorbency in an acidic environment. When fully hydrated, the absorbent medical device is substantially transparent.

This is advantageous in wound care applications since the state of the underlying wound can be determined without removing the dressing. This renders them ideal for use as an absorbent wound care product, such as a dressing, or as part of an absorbent wound care product. They are suited for use in wound dressings for acute wounds, including surgical wounds, and chronic and burn wounds.

They are particularly useful for wounds with moderate to high levels of exudates, and for flat or cavity wounds of this type. Typical examples include burn wounds, and chronic wounds, such as pressure sores and leg ulcers. The dressing, when covering a wound, is able to prevent water in body fluids from being lost, providing a favourable moist environment for wound healing and maintaining a fluid-free, maceration-free, germ-free wound surface.

They may also be used in ostomy devices, and surgical and dental sponges.

Thus, in one embodiment of this aspect of the present invention, an absorbent medical device comprising the derivatised chitosan fibres is provided.

Preferred derivatised chitosan products for use in wound care medical devices are carded, needle-bonded non-wovens.

The use of the absorbent materials of the present invention is not limited to medical products, however, and they are useful for other applications.

Their absorbent properties, biodegradability, and the fact that chitosan is a renewable material, mean that absorbent devices comprising the derivatised chitosans of the invention are also particularly desirable for use in absorbent pads for the personal care sector, particularly for disposable sanitary devices such as nappies (diapers), disposable nappies and training pants, feminine care products, for example, tampons, sanitary towels, or napkins and pant liners, and incontinence products.

Thus, in another embodiment of this aspect of the present invention, an absorbent personal care device comprising the derivatised chitosan fibres is provided.

In a fourth aspect the invention provides a method of use of an absorbent device according to the present invention. In one embodiment, it provides a method of treatment of the human or animal body using an absorbent device according to the present invention, in particular in wound care.

The invention will now be illustrated by the following non-limiting examples.

### EXAMPLE 1

This example demonstrates the process for making super-absorbent gelling, water insoluble modified chitosan fibres by carboxymethylation of the fibres.

A solution containing 10.625 kg sodium hydroxide (40% solution), 4.5 kg sodium chloroacetate (44% solution) and 100 kg ethanol (60% solution) was prepared, the remainder of the solutions being deionised water. The solution was heated to 30° C. 5.0 kg chitosan fibres were added and the solution reacted for 45 minutes. The temperature of the solution was then increased to 40° C., and the fibres were reacted for a further 120 minutes. The modified fibres underwent 5 spin dry and ethanol wash cycles and a final spin dry. The fibres were treated with a spin finish comprising 1.523% Tween 20 and 98.478% ethanol (95%). The fibres underwent another spin dry after fibre finish application and were left to air dry and condition. Fibres which are untreated with Tween 20 retain good hydrophilicity during processing.

### EXAMPLE 2

The product fibres of Example 1 were processed according to known methods on a non-woven processing machine into a non-woven fabric.

The gram per gram absorbency of the fabric was determined as follows:

Three fabric specimens were cut to 5 cm×5 cm (2″ by 2″) (25 cm²). Each specimen was weighed. Each specimen was then placed in a Petri dish and covered with an excess of Solution A. Solution A is a specific solution of sodium chloride and calcium chloride QCP090 as used in British/European Standard BS EN 13726-1:2002 Test methods for primary wound dressings Part 1 Aspect of absorbency.)

Each specimen was left in the Solution A for 180 seconds then removed from the Solution A by taking one corner with the forceps, and allowed to drain for 15 seconds. Each specimen was then weighed.

The absorbency of the fabric was 18.2 g/g

1. A process for producing a salt of a chitosan acid anion derivative, which comprises contacting chitosan fibre
(a) with a base in an aqueous or non-aqueous medium and
(b) with a solution in an aqueous or non-aqueous medium of a salt of the formula (I):
M-R-T (I)
wherein
M is a Group IA metal cation;
R is a hydrocarbyl oxoacid anion, substituted by T; and
T is a nucleophilic leaving group,
all at a temperature below 50° C.

2. A process according to claim 1, which is carried out at a temperature in the range of 25 to 45° C.

3. A process according to claim 1, which is carried out at a temperature in the range of 30 to 40° C.

4. A process according to claim 1, in which the base is sodium hydroxide with a percentage concentration with respect to the total reaction mixture of 2.5 to 10 w/v %.

5. A process according to claim 1, which is a one-step process.

6. A process according to claim 1, which is carried out in a mixture of water and ethanol.

7. A process according to claim 1, in which the chitosan is in the form of fibre tow and has a degree of chitin deacetylation of from 60 to 100% and an average molecular weight between 3800 to 20,000 daltons.

8. A process according to claim 1, in which M is a sodium cation.

9. A process according to claim 1, in which R is acetate, substituted by a chloro group in the 2-position.

10. A process according to claim 1, in which R is methanesulphonate, substituted by a nucleophilic leaving group T in the 1-position.

11. A salified hydroxyl (inorganic or organic acyl) hydrocarbyl substituted chitosan product of the process according to claim 1.

12. A product according to claim 10, wherein the average degree of substitution is from about 0.2 to 0.3.

13. A product according to claim 10, having an absorbency of 16 to 23 g/g.

14. A non-woven fabric comprising a fibrous product according to claim 11.

15. A non-woven fabric according to claim 14 wherein the fibrous product is the only absorbent material present.

16. A non-woven fabric according to claim 14 which is reinforced with a reinforcing fibre blended with the water-insoluble derivatised chitosan.

17. A non-woven fabric according to claim 14 which comprises unmodified chitosan in a weight ratio of chitosan fibre to the product chitosan fibre of 1:3 to 3:1.

18. An absorbent device comprising a non-woven fabric according to claim 14.

19. A method of treatment of the human or animal body using an absorbent device according to claim 18 in wound care.
